(19) United States
(12) Patent Application Publication
(10) Pub. No.: US 2008/0262579 A1
(43) Pub. Date: Oct. 23, 2008

(54) Exercise method and apparatus which provides therapeutic modalities and reflexology

(76) Inventors: Kevin J. Berk, Paradise Valley, AZ (US); Je're Taffet, Paradise Valley, AZ (US)

Correspondence Address: Greg L. Martinez, Suite 408, 3116 South Mill Avenue, Tempe, AZ, 85282, US

(21) Appl. No.: 11/404,626

(22) Filed: Apr. 14, 2006

(51) Int. Cl. A61F 7/08

(52) U.S. Cl. 607/104

(57) ABSTRACT

An exercise apparatus includes a thermal system which carries a support structure. The support structure includes an exercise mat. A conduit extends through the exercise mat and has acupressure structures connected to opposed ends. The acupressure structures provide acupressure to a person exercising on the mat. The thermal system flows heat to and from the conduits and acupressure structures to provide the person with therapeutic modalities.

### BACKGROUND OF THE INVENTION

1. Field of the Invention

This invention relates generally to exercising and, more particularly, to exercising equipment.

2. Description of the Related Art

Therapeutic modalities have been used in the art to enhance the emotional and physical well-being of a person. Therapeutic modalities generally include hot therapy, cold therapy, alternating hot and cold therapy, and massaging. Alternating hot and cold therapy is often referred to as contrast therapy. A review of therapeutic modalities can be found in U.S. Patent Application No. 20030204227 titled “Therapeutic hot/cold pad”. Several references describe apparatus useful for these purposes. For example, U.S. Pat. No. 6,206,463, and the references included therein, disclose seats and U.S. Pat. No. 6,918,144 discloses a self-heating portable massage table.

It has also been known in the art to use reflexology to enhance the emotional and physical well-being of the person. Reflexology generally involves a method of massage that relieves tension through the application of pressure to the person. This pressure can be applied to various parts of the person's body, such as the anterior, posterior, feet, and hands. There are several references which disclose apparatus for providing reflexology. For example, an acupressure pad is described in U.S. Pat. No. 4,974,582 and an acupressure foot massage mat is described in U.S. Pat. No. 5,158,073.

These references for therapeutic modalities and reflexology each include apparatus useful for their intended purpose. However, the apparatus disclosed do not provide therapeutic modalities as well as acupressure. Further, they are also not useful for a person to exercise on. For example, the apparatus disclosed in U.S. Pat. Nos. 6,206,463 and 6,918,144 are for use as a seat and table, respectively. The acupressure pad described in U.S. Pat. No. 4,974,582 is useful for a person to lie on. The acupressure foot massage mat disclosed in U.S. Pat. No. 5,158,073 is useful for a person to walk slowly on, but is not intended for more strenuous exercising or for providing therapeutic modalities. Accordingly, there is a need in the art for an exercise apparatus which is useful for therapeutic modalities and reflexology.

### BRIEF SUMMARY OF THE INVENTION

The invention employs an exercise apparatus which provides both therapeutic modalities and acupressure to the person while he or she is supported on it. The exercise apparatus includes a thermal system that carries a support structure. When the person is supported on the support structure, he or she can be performing many different types of exercises. Some examples include yoga, Pilates, stretching, That yoga, aerobics, walking, and martial arts, among others. In one embodiment, the support structure includes an exercise mat with a conduit extending therethrough. In some of these embodiments, one or more acupressure structures are connected to the conduit. The conduit and acupressure structure(s), if included, provide acupressure to a person supported on the support structure. The thermal system flows heat to and from the conduits and acupressure structure(s) to provide therapeutic modalities through the support structure to the person supported thereon. The mat is preferably made of a low thermal conductivity material and the conduit and acupressure structure(s) are preferably made of a high thermal conductivity material so that the therapeutic modalities and acupressure are provided in the same region.

These and other features, aspects, and advantages of the present invention will become better understood with reference to the following drawings, description, and claims.

### BRIEF DESCRIPTION OF THE DRAWINGS

FIGS. 1a and 1b are side views of a support structure, in accordance with the present invention, which includes a mat and conduits extending therethrough.

FIG. 1c is a side view of another embodiment of a support structure, in accordance with the present invention.

FIG. 2a is a side view of an exercise apparatus, in accordance with the present invention, which includes a thermal system and the support structure of FIGS. 1a and 1b.

FIG. 2b is a side view of the exercise apparatus of FIG. 2a with acupressure structures on the mat, in accordance with the present invention.

FIG. 2c is a side view of the exercise apparatus of FIG. 2b with acupressure structures on the mat and between the mat and thermal system, in accordance with the present invention.

FIG. 2d is a side view of the exercise apparatus of FIG. 2b with acupressure structures on the mat and between the mat and thermal system, in accordance with the present invention.

FIG. 2e is a side view of another embodiment of an exercise apparatus, in accordance with the present invention.

FIG. 2f is a side view of the acupressure structures used in the exercise apparatus of FIG. 2e.

FIG. 3 is a perspective view of acupressure structures and the conduit, in accordance with the present invention.

FIGS. 4a and 4b are perspective and side views, respectively, of another embodiment of an exercise apparatus, in accordance with the present invention.

FIG. 4c is a top view of the thermal system included in the exercise apparatus of FIGS. 4a and 4b.

FIG. 4d is a side view of another embodiment of an exercise apparatus, in accordance with the present invention, having separate pockets for holding acupressure structures.

FIGS. 5a and 5b are side and top views, respectively, of an embodiment of a thermal system, in accordance with the present invention, which includes trenches.

FIG. 5c is a top view of another embodiment of the thermal system of FIGS. 5a and 5b, in accordance with the present invention.

FIGS. 5d and 5e are top views of another embodiment of the thermal system of FIG. 5a, in accordance with the present invention.

FIG. 6 is a perspective view of another embodiment of a thermal system, in accordance with the present invention, which includes compartments.

FIG. 7 is a block diagram of a system which can be used to provide therapeutic modalities for the person exercising on an exercise apparatus, in accordance with the present invention.

FIG. 8 is a flowchart of a method of exercising using an exercise apparatus, in accordance with the present invention.

FIG. 9 is a flowchart of a method of fabricating an exercise apparatus, in accordance with the present invention.

### DETAILED DESCRIPTION OF THE INVENTION

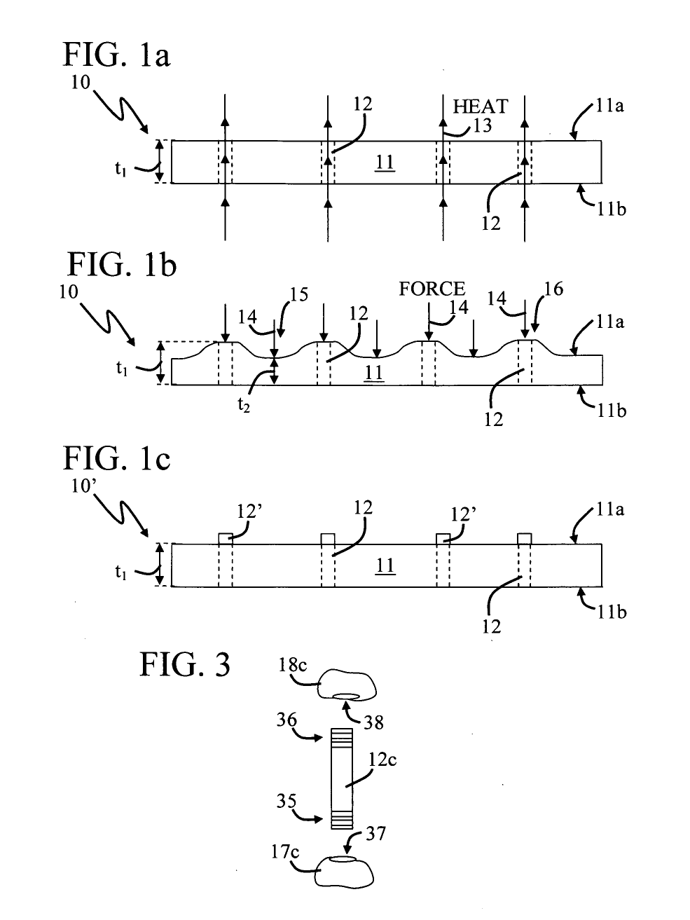

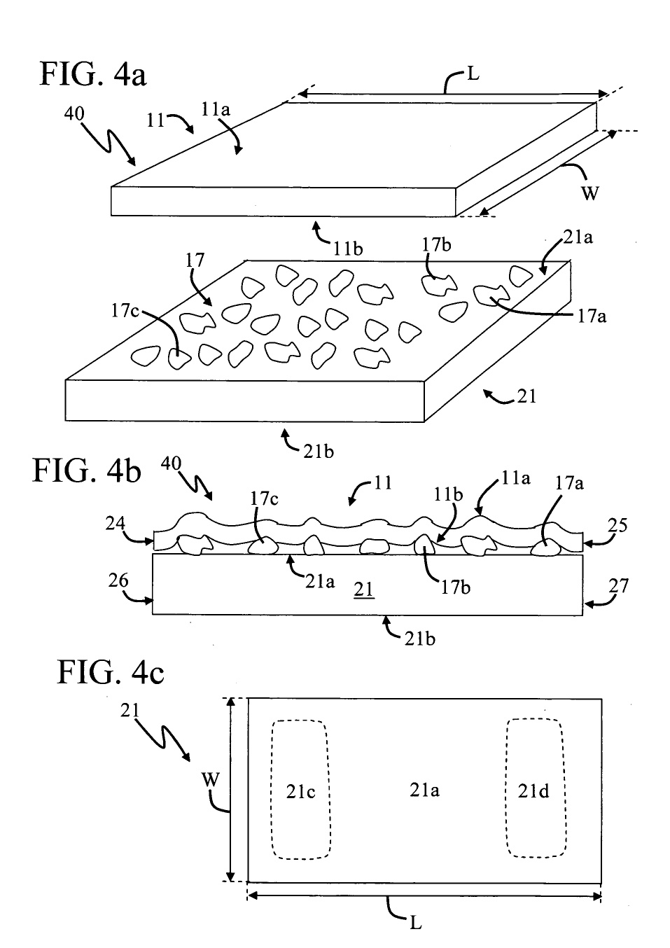

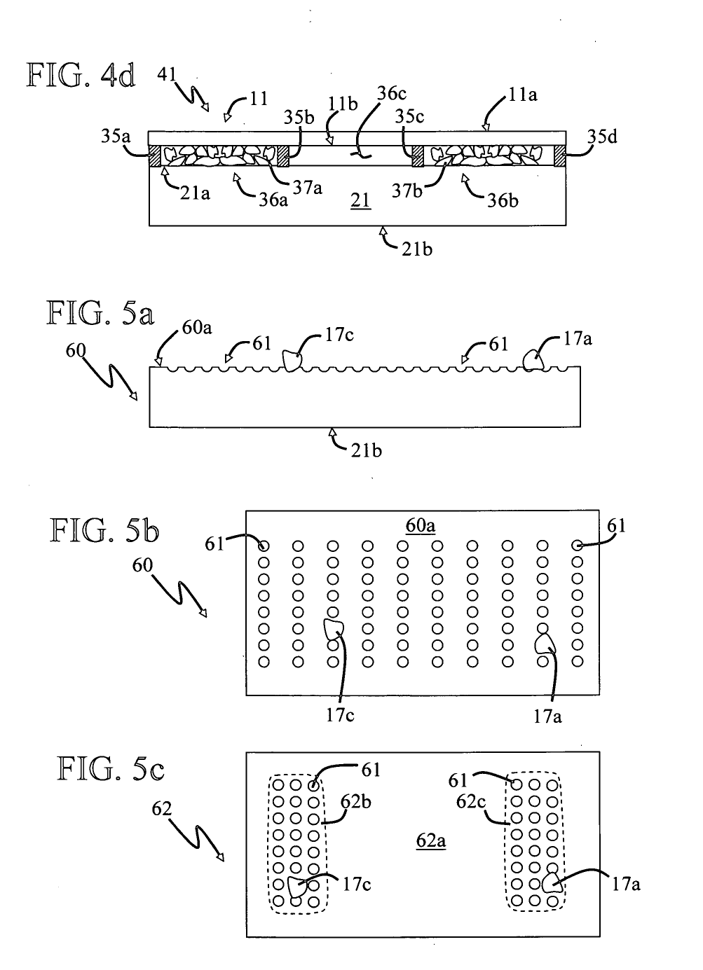

FIGS. 1a and 1b are side views of a support structure 10, in accordance with the present invention. In this embodiment, support structure 10 includes an exercise mat 11 having a thickness t₁ when a person is not supported on it. Support structure 10 can be rolled and unrolled so that it is easier and more convenient to store and transport. Support structure 10 is dimensioned so that the person can comfortably lie down on it, such as when stretching. A preferred dimension is a width W of about 3 feet and a length L of about 6 feet (FIG. 4a), although it can have other dimensions. For example, if mat 11 is to be used for standing on, such as during aerobics, then it can have smaller dimensions. Mat 11 can have larger dimensions if it is intended to be used by more than one person at the same time, such as during an exercise class.

Exercise mat 11 is preferably made of a low thermally conductive material, which is also resilient. Examples of such materials include rubber, plastic, foam, and cloth, but there are others that can also be used. These materials typically have a low thermal conductivity coefficient below about 0.7 Watts (W) per meter (m) per Kelvin (K) (W/(m×K)). Most low thermally conductive materials generally have a thermal conductivity coefficient between about 0.7 W/(m×K) and 0.001 W/(m×K). There are many references which provide the thermal conductivity coefficients of materials. One reference is University Physics, 7th Ed. Table 15-5, by Hugh D. Young.

In accordance with the invention, support structure 10 also includes conduits 12 extending through mat 11 between an exercise surface 11a and an opposed surface 11b. As discussed in more detail below, conduits 12 include a high thermal conductivity material so that they operate as a conduit for the flow of heat through mat 11. The material included in conduits 12 is also harder than that included in mat 11 so that conduits 12 also provide acupressure to the person supported on surface 11a.

Examples of these materials include stone and metal. Some types of stone that can be used include basalt, marble, shale, and granite, and some types of metals that can be used include steel and aluminum. Most stones have a thermal conductivity coefficient of about 2 W/(m×K) to about 5 W/(m×K) and most metals have a thermal conductivity coefficient of about 50 W/(m×K) to about 450 W/(m×K). Hence, a high thermally conductive material typically has a thermal conductivity coefficient between about 2 W/(m×K) and 450 W/(m×K). It should be noted that conduits 12 preferably have a thermal conductivity coefficient that is greater than three times that of mat 11. This is so that heat will flow mostly through conduits 12 instead of mat 11. This is desirable so that the hot and cold therapy is provided at or near where conduits 12 provide acupressure to the person.

In this example, a plurality of conduits 12 extends through mat 11. However, in general, one or more conduits can be included. More heat flows between surfaces 11a and 11b as the number of conduits 12 extending through mat 11 increases. Less heat flows between surfaces 11a and 11b as the number of conduits 12 extending through mat 12 decreases. Hence, the number of conduits 12 that extend through mat 11 can be chosen to provide a desired amount of heat flow through mat 11.

In some embodiments, mat 11 can include a high thermally conductive filler material, such as metal or stones, which increases the flow of heat through it by increasing its thermal conductivity. For example, mat 11 can include rubber or plastic impregnated with metal or stone. The flow of heat through mat 11 increases as the amount of this filler material included therein increases and the flow of heat through mat 11 decreases as the amount of this filler material included therein decreases. In this way, the amount of heat that flows through mat 11 can be controlled by controlling the amount of filler material included in it.

In this example, heat 13 is shown flowing from surface 11b to surface 11a through conduit 12 to provide heat therapy to the person while supported on surface 11a. In this way, the flow of heat 13 increases the temperature of conduit 12 and provides it with a desired high temperature. In another example, however, heat 13 can flow through conduit 12 from surface 11a to surface 11b to provide cold therapy to the person while exercising on surface 11a. In this way, the flow of heat 13 decreases the temperature of conduit 12 and provides it with a desired low temperature. Hence, in accordance with the invention, support structure 10 provides therapeutic modalities to the person while he or she is supported on it. As will be discussed in more detail below, the flow of heat 13 can be controlled in many different ways, such as with a thermal system.

In accordance with the invention, support structure 10 also provides reflexology to the person while he or she is supported on it. One way support structure 10 does this is shown in FIG. 1b. In this example, support structure 10 provides acupressure by changing its shape in response to a force 14 applied to it. In particular, portions of surface 11a move towards surface 11b in response to force 14 being applied thereto. Force 14 is typically provided by the person being supported on surface 11a. Hence, force 14 can be, for example, from the person's anterior, posterior, hands, feet engaging surface 11a.

The value of force 14 depends on many different factors, such as the person's weight. Force 14 is generally larger when the person performs more strenuous exercises while supported on structure 10 and smaller when the person performs less strenuous exercises. Force 14 can also depend on the type of exercises the person performs while supported on structure 10. For example, force 14 is typically larger when the person is performing aerobics and smaller when the person is stretching.

In this example, if force 14 is applied to surface 11a near conduit 12, surface 11a does not substantially move towards surface 11b. This is because materials with high thermal conductivities tend to be harder than materials with low thermal conductivities. Hence, if force 14 is applied to surface 11a away from conduit 12, surface 11a moves towards surface 11b. This is because mat 11 is made of a low thermal conductivity material, as discussed above, which tends to be softer than high thermal conductivity material. When force 14 is applied to surface 11a, a region 16 near conduit 12 has thickness t₁ and a region 15 between adjacent conduits 12 has a thickness t₂, wherein thickness t₁ is greater than thickness t₂.

In other examples, thickness t₁ in region 16 can decrease in response to force 14, but it is preferably still greater than thickness t₂ in region 15. It should be noted that if mat 11 includes filler material, then the difference between thicknesses t₁ and t₂ in response to force 14 is generally smaller, but still large enough so that conduit 12 provides acupressure. In this way, support structure 10 provides acupressure to the person while he or she is supported on surface 11a. As will be discussed presently in FIG. 1c, however, support structure 10 can provide acupressure and therapeutic modalities in many other different ways.

FIG. 1c is a side view of a support structure 10′, in accordance with the invention. Support structure 10′ includes mat 11 having conduits 12 extending therethrough. In this embodiment, however, a portion 12′ of conduit 12 extends above surface 11a. In this way, portion 12′ provides acupressure and therapeutic modalities to a person supported by support structure 10′. Portion 12′ can have many different shapes and is shown here as being square for simplicity. However, in other examples, it can have another shape, such as rectangular, curved, or round.

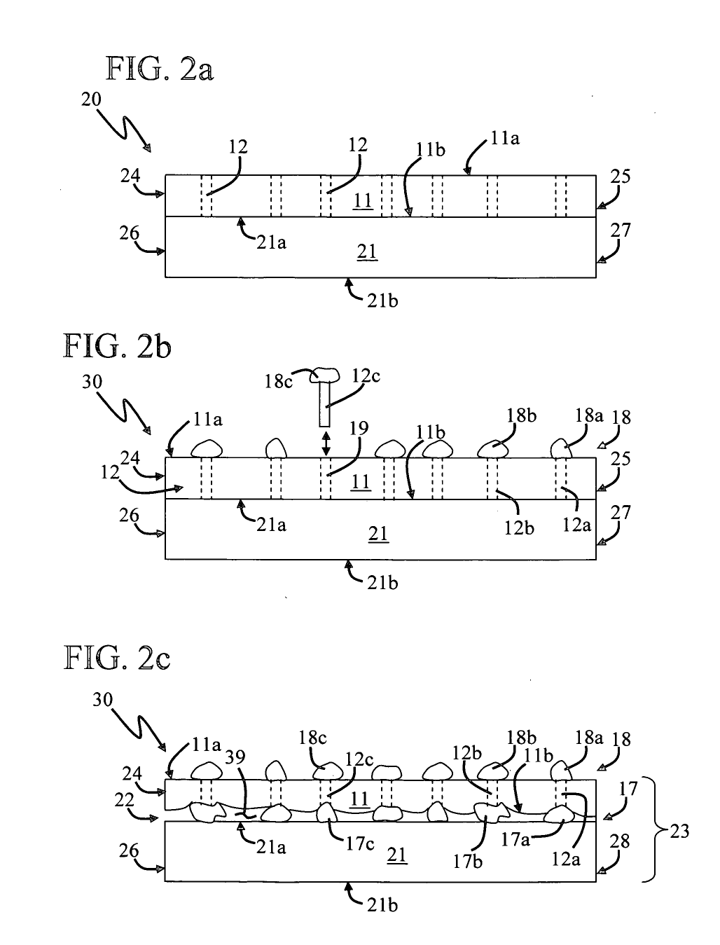

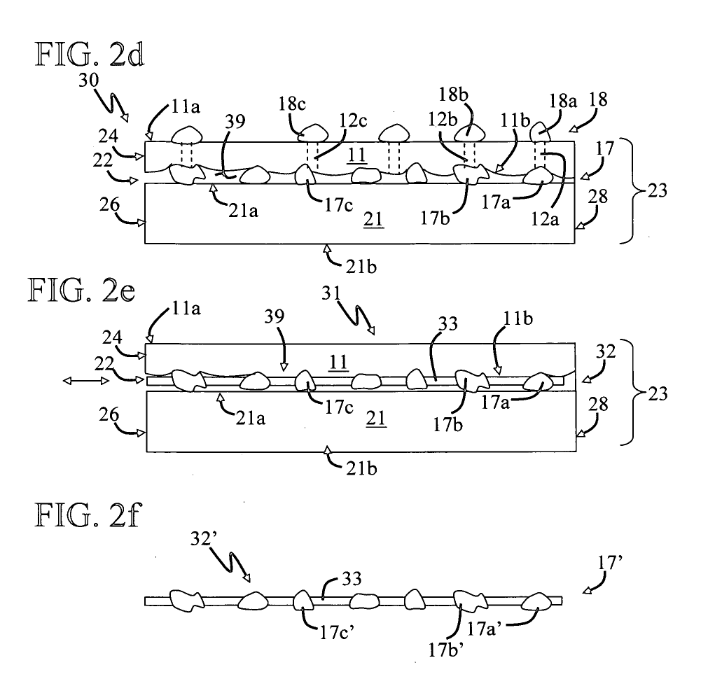

FIG. 2a is a side view of an apparatus 20 for exercising, in accordance with the present invention. Apparatus 20 includes a thermal system 21 which carries support structure 10. Thermal system 21 controls the flow of heat 13 (FIG. 1a) and operates as a heater/cooler so it can provide contrast therapy to the person supported on surface 11a. In other examples, thermal system 21 can operate as a heater or cooler, but here it operates as both for illustrative purposes. Hence, in other examples, thermal system 21 can be replaced with a heater or cooler to provide only hot or cold therapy, respectively.

Thermal system 21 is preferably small and portable so it can be easily moved around and stored. System 21 can be powered in many different ways. It is preferably powered by connecting it to an electrical outlet. However, in other examples, it can be powered by a battery system or solar energy.

Surface 11b of mat 11 is positioned adjacent to a surface 21a of thermal system 21. An opposed surface 21b of thermal system 21 is for engaging a support structure (not shown), such as a floor. Thermal system 21 can have the same length L and width W as exercise mat 11 (FIG. 4c), but in other examples it can have different length and/or width dimensions. For example, if the length and width of system 21 is larger than that of mat 11, then more heat flows between it and surface 11a.

Support structure 10 is positioned on thermal system 21 so that heat 13 flows between system 21 and conduits 12. The flow of heat 13 to and from thermal system 21 depends substantially on the material included in mat 11 and conduits 12. As the thermal conductivity of the material in conduits 12 increases and that of the material in mat 11 stays the same, more heat flows through conduits 12 and less heat flows through mat 11. As the thermal conductivity of the material in conduits 12 decreases and that of the material in mat 11 stays the same, less heat flows through conduits 12 and more heat flows through mat 11. Further, as the thermal conductivity of the material in conduits 12 stays the same and that of the material in mat 11 increases, less heat flows through conduits 12 and more heat flows through mat 11. As the thermal conductivity of the material in conduits 12 stays the same and that of the material in mat 11 decreases, more heat flows through conduits 12 and less heat flows through mat 11.

In accordance with the invention, since thermal system 21 operates as a heater/cooler, it has one mode of operation in which it operates as a heater and another mode of operation in which it operates as a cooler. Thermal system 21 can switch between these two modes of operation to provide contrast therapy. In both of these modes, thermal system 21 operates to control the temperature of conduits 12. When thermal system 21 operates as a heater, it provides heat 13 which flows through conduit 12 from surface 11b to surface 11a. In this way, conduits 12 provide heat therapy as well as acupressure to the person supported on surface 11a. When thermal system 21 operates as a cooler, heat 13 flows through conduit 12 from surface 11a to surface 11b, and to thermal system 21. In this case, the flow of heat 13 away from conduits 12 has a cooling affect on them. In this way, conduits 12 provide cold therapy, as well as acupressure, to the person supported on surface 11a. Hence, apparatus 20 provides contrast therapy as well as acupressure. Further, apparatus 20 provides the contrast therapy and acupressure to the person in the same region of the person's body.

Thermal system 21 can include many different types of heaters and coolers to provide contrast therapy. For cold therapy, it can include a thermoelectric cooler. For heat therapy, it can include an electrical wire which operates as a filament and heats up in response to an electrical signal flowing through it, as in a thermoelectric heater. One type of thermoelectric heater is used in heating pads for treating injuries.

More specific examples of heater and coolers can be found in several references. For example, U.S. Pat. No. 4,523,594 discloses a stretchable textile heat-exchange jacket and U.S. Pat. No. 4,962,761 discloses a thermal bandage. U.S. Pat. No. 4,459,468 discloses a temperature control fluid circulating system and U.S. Pat. No. 4,170,998 discloses a portable cooling apparatus. U.S. Pat. No. 4,094,357 discloses a heat transfer blanket and U.S. Pat. No. 3,967,627 discloses a hot/cold applicator system. U.S. Pat. No. 3,888,259 discloses a hypothermia system. U.S. Pat. No. 3,154,926 discloses a cooling blanket and U.S. Pat. No. 4,981,135 discloses a therapeutic thermal cuff. These examples can be modified to operate with apparatus 20 as thermal system 21 to flow heat 13 to and from conduits 12 as described above.

In this embodiment, thermal system 21 and mat 11 are separate pieces. Mat 11 includes opposed sidewalls 24 and 25 which extend between surfaces 11a and 11b and thermal system 21 includes opposed sidewalls 26 and 27 which extend between surfaces 21a and 21b. Since mat 11 and thermal system 21 are separate pieces, sidewall 24 is discontinuous with sidewall 26 and sidewall 25 is discontinuous with sidewall 27. In other embodiments, however, thermal system 21 and mat 11 can be integrated together in a single piece 23 (FIGS. 2c and 2d). For example, thermal system 21 and mat 11 can be fixedly attached together with a fastener, such as glue or screws, or thermal system 21 can be positioned inside of mat 11. In different embodiments of piece 23, sidewall 24 is continuous with sidewall 26 and/or sidewall 25 is continuous with sidewall 27.

FIG. 2b is a side view of an apparatus 30 for exercising, in accordance with the present invention. Apparatus 30 is similar to apparatus 20 described above, except it includes acupressure structures 18 attached to conduits 12 near surface 11a. In this example, acupressure structures 18 include separate acupressure structures, some of which are denoted as 18a, 18b, and 18c. Acupressure structures 18a, 18b, and 18c are connected to conduits 12a, 12b, and 12c, respectively. Conduits 12a, 12b, and 12c are the same as conduits 12 discussed above. Acupressure structures 18a, 18b, and 18c can include the same or similar material as conduits 12a, 12b, and 12c, and preferably include high thermally conductive material. In other examples, however, acupressure structures 18 include a low thermally conductive material.

In this embodiment, acupressure structures 18a, 18b, and 18c include stones and are shaped to provide acupressure to the person using apparatus 10. Structures 18a, 18b, and 18c can also include sand, beads, ball bearings, etc. In some examples, the stones are flat and shaped similar to the contours of the human body. In other examples, the stones are shaped so they extend from surface 11a and the person can balance on them with his or her hands above surface 11a. In operation, heat 13 flows in a manner similar to that described above. Here, however, heat 13 flows through acupressure structures 18a, 18b, and 18c and corresponding conduits 12a, 12b, and 12c. In this way, thermal system 21, acupressure structures 18a, 18b, 18c, and conduits 12a, 12b, 12c provide therapeutic modalities and acupressure to the person while he or she is supported on apparatus 30.

As shown in FIG. 2b, acupressure structures 18a, 18b, and 18c, and corresponding conduits 12a, 12b, and 12c, are repeatably moveable between engaged and disengaged positions relative to mat 11. To illustrate this feature more clearly, conduit 12c and acupressure structure 18c are shown in the disengaged position relative to mat 11. In particular, conduit 12c and acupressure structure 18c are shown away from an opening 19 that extends through mat 11 between surfaces 11a and 11b. When in the engaged position, conduit 12c is received by opening 19 and acupressure structure 18c engages surface 11a. Conduit 12c preferably contacts thermal system 21 when it is in the engaged position so that more heat can flow therebetween. In FIG. 2b, conduit 12a and corresponding acupressure structure 18a, as well as conduit 12b and corresponding acupressure structure 18b, are shown in engaged positions relative to mat 12.

It should be noted that in some examples, acupressure structures 17 can be attached to conduits 12 near surface 11b. This feature is shown in FIG. 2c, which is a side view of apparatus 30. Acupressure structures 17 can be the same or similar to acupressure structures 18 discussed above. In this example, acupressure structures 17 includes separate acupressure structures, some of which are denoted as 17a, 17b, and 17c. Acupressure structures 17a, 17b, and 17c are connected to corresponding conduits 12a, 12b, and 12c opposite acupressure structures 18a, 18b, and 18c, respectively. In this way, acupressure structures 18a, 18b, and 18c engage surface 11a and acupressure structures 17a, 17b, and 17c engage surface 11b when they are in the engaged position relative to mat 11. By having acupressure structures 17a, 17b, 17c and 18a, 18b, 18c on opposite ends of corresponding conduits 12a, 12b, and 12c, conduits 12a, 12b, and 12c are more securely held to mat 11.

Also in FIG. 2c, mat 11 and thermal system 21 are integrated together to form single piece 23 with a cavity 39 formed between surfaces 11b and 21a. An opening 25 extends between surfaces 11b and 21a near sidewalls 24 and 26 and into cavity 39. This allows acupressure structures 17 to be inserted into and removed from cavity 39. In piece 23, sidewalls 25 and 27 are connected together and form a sidewall 28. Sidewall 28 is preferably a continuous sidewall, but it can be discontinuous, such as when the length L and width W dimensions of mat 11 and thermal system 21 are not the same. In some examples, a fastener, such as a zipper or buttons, can be positioned at or near opening 25 so that it can be repeatably opened and closed as desired. Opening 25 is generally opened to insert acupressure structures 17 into or remove them from cavity 39. When acupressure structures 17 are inserted in cavity 39, opening 25 is preferably closed by the fastener to hold structures 17 therein.

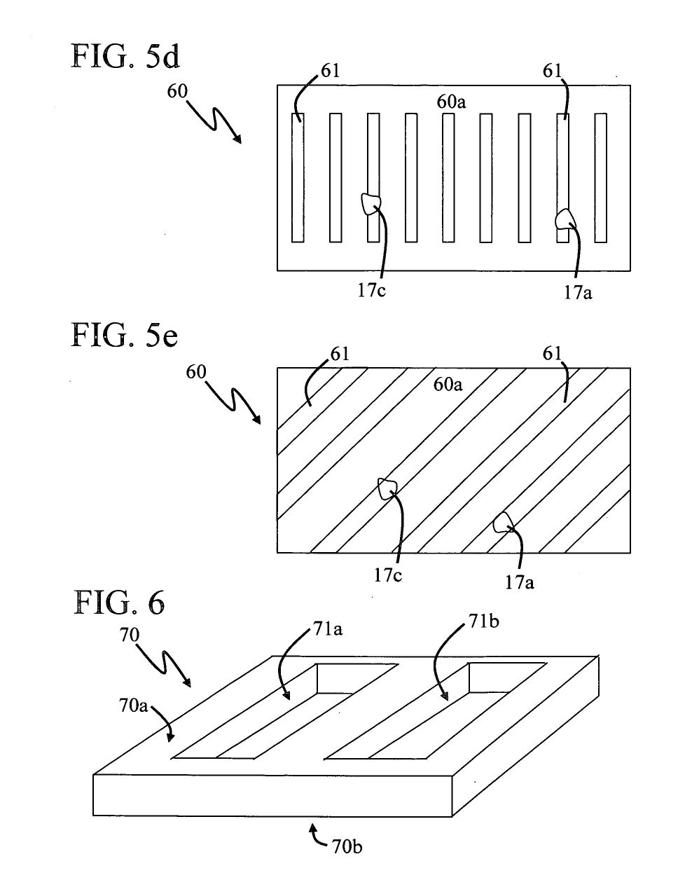

In some examples, some of structures 17 are not connected to conduits 12. This feature is shown in FIG. 2d, which is a side view of apparatus 30. Here, acupressure structure 18c is attached to conduit 12c near surface 11a, but acupressure structure 17c is not attached to conduit 12c near surface 11b. In this way, acupressure structure 17c is loosely held in cavity 39 so that it can move around therein. This is useful so that acupressure structure 17c can be positioned at a desired location along surface 11b, as will be discussed in more detail with FIGS. 5a-5c, to provide acupressure and therapeutic modalities to a desired location relative to the person's body. In other examples, acupressure structure 17c is attached to conduit 12c, but acupressure structure 18c is not so that it is loosely positioned on surface 11a.

In some examples, acupressure structures 17 and/or 18 are repeatably moveable between engaged and disengaged positions to and from corresponding conduits 12. In other examples, structures 17 and/or 18 are integrated with conduits 12 so that they form a single piece. Acupressure structures 17 and 18 can be repeatably moveable to and from conduits 12 in many different ways, such as with a threaded fastener. If conduits 12 and acupressure structures 17 and 18 are integrated together, they can be formed in many different ways, such as with conventional stone or metal cutting techniques, depending on the material they are made of.

FIG. 3 shows one example of how conduit 12c can be attached to acupressure structures 17c and 18c so that they are repeatably moveable between engaged and disengaged positions relative to each other. In some examples, conduit 12c and acupressure structures 17c and 18c can include the same material, such as metal and stone, or various combinations thereof. Here, conduit 12c is metal and includes threaded ends 35 and 36. Further, acupressure structures 17c and 18c are stone and include threaded openings 37 and 38, respectively. In FIG. 3, structures 17c and 18c are shown in the disengaged position relative to conduit 12c. Threaded openings 37 and 38 are sized and shaped to receive threaded ends 35 and 36, respectively, so that structures 17c and 18c can be engaged to conduit 12c, as shown in FIG. 2c. FIG. 2d shows structure 17c disengaged from conduit 12c and structure 18c engaged to it.

FIG. 2e is a side view of another embodiment of an exercise apparatus 31, in accordance with the present invention. In this embodiment, acupressure structures 17 are connected to a support structure 33 so that structures 17 and 33 are a single integrated piece 32. This is advantageous so that piece 32 can be repeatably inserted into and removed from cavity 39. Piece 32 can be replaced with another similar piece, such as piece 32′ shown in FIG. 2f, having different types of acupressure structures. For example, the different acupressure structures can have different dimensions, shapes, and/or include different materials. In this particular embodiment, piece 32′ includes acupressure structures 17′ which are smaller than structures 17, have the same shape, and include the same material. For example, structures 17a′, 17b′, and 17c′ are smaller than structures 17a, 17b, and 17c, respectively.

FIGS. 4a and 4b are perspective and side views, respectively, of an exercise apparatus 40, in accordance with the present invention. In this embodiment, apparatus 40 includes mat 11 carried by thermal system 21. FIG. 4c is a top view of thermal system 21 included in apparatus 40 of FIGS. 4a and 4b. Acupressure structures 17, some of which are denoted as 17a, 17b, and 17c, are carried loosely between surfaces 11b and 21a. In this embodiment, thermal system 21 operates as described above and acupressure structures 17 provide acupressure through mat 11 for the person exercising on surface 11a. In some embodiments, acupressure structures 18a, 18b, and 18c can be carried loosely on surface 11a.

An advantage of acupressure structures 17a, 17b, and 17c being loosely carried on surface 21a is that they can be positioned at desired locations thereon. For example, it may be desirable to position some or all of acupressure structures 17 in a region 21c (FIG. 4c), which corresponds to a location under the person's head when he or she is lying on mat 11. It may also be desirable to position some or all of acupressure structures 17 in a region 21d, which corresponds to a location under the person's feet when he or she is lying on mat 11. In this way, the person can control the amount of acupressure and heat provided to different portions of his or her body.

It should be noted that acupressure structures 17 are optional in apparatus 40. For example, in some embodiments, mat 11 is carried by thermal system 21 so that surface 11b engages surface 21a. In still other embodiments, thermal system 21 is positioned within mat 11. In these embodiments, mat 11 is preferably made of a hard resilient material, such as plastic and rubber, so that thermal system 21 is less likely to be damaged when the person is exercising on surface 11a. For example, if thermal system 21 includes filaments which provide the heat, then mat 11 will better protect the filaments from breaking if it includes a hard resilient material. Another advantage is that the hard resilient material makes apparatus 40 more durable so it has a longer lifetime. Further, the hard resilient material makes apparatus 40 more sturdy so the person can exercise on it in a safer manner.

In other embodiments, apparatus 40 includes acupressure structures which have a low thermal conductivity and are attached to mat 11 or integrated therewith. In these embodiments, thickness t₁ of mat 11 is preferably chosen so that a desired amount of heat flows through it.

FIG. 4d is a side view of an embodiment of an exercise apparatus 41, in accordance with the present invention. In this embodiment, apparatus 41 is similar to apparatus 40 described above. One difference, however, is that mat 11 is fixedly attached to thermal system 21 through sidewalls 35a, 35b, 35c, and 35d. Sidewalls 35a and 35d extend between mat 11 and thermal system 21 along their outer periphery. Sidewall 35b is spaced apart from sidewall 35a so that they bound a cavity 36a. Sidewall 35c is spaced apart from sidewall 35d so that they bound a cavity 35b. Similarly, sidewalls 35b and 35c are spaced apart so that they bound a cavity 35c, which is between cavities 35a and 35b. It should be noted that cavities 36a, 36b, and 36c are also bounded by surfaces 11b and 21a of mat 11 and thermal system 21, respectively.

In accordance with the invention, cavities 36a, 36b, and 36c hold acupressure structures. The acupressure structures can be the same or similar to structures 17 and 18 described above. In this embodiment, cavities 36a and 36b hold acupressure structures 37a and 37b, respectively. Further, cavity 36b is empty, although in other examples, it can also hold acupressure structures. Here, structures 37a and 37b include a plurality of stones, but they can include other materials, such as sand, pebbles, metal pieces, etc., which may or may not conduct heat

Structures 37a and 37b are loosely held in cavities 36a and 36b so that they can move in response to a force applied to mat 11 and conform to the shape of the object providing the force. In one particular example, the force applied to acupressure structures 36a is from the person's head and the force applied to acupressure structures 36b is from the person's feet.

FIGS. 5a and 5b are side and top views, respectively, of an embodiment of a thermal system 60, in accordance with the present invention. Thermal system 60 includes an upper surface 60a which has a plurality of trenches 61 extending therethrough. Trenches 61 can have many different shapes, but are preferably cup-shaped. They are also dimensioned so that the movement of acupressure structures 17, such as structures 17a and 17c, is restricted along surface 60a (FIG. 5b). In this way, structures 17a and 17c are more likely to be held in desired locations on surface 60a. Also, mat 11 is less likely to move relative to surface 60a if structures 17a and 17c are connected to conduits 12a and 12c, respectively, as shown in FIG. 2c. Trenches 61 can have other shapes and dimensions, which are preferably chosen based on the shape and dimensions of structures 17.

FIG. 5c is a top view of an embodiment of a thermal system 62, in accordance with the present invention. System 62 is similar to thermal system 60 discussed above. One difference, however, is that trenches 61 are positioned at desired locations on an upper surface 62a of thermal system 62. In this embodiment, trenches 61 are located in a region 62b, which corresponds to a location under the person's head when he or she is lying on mat 11 and a region 62c, which corresponds to a location under the person's feet when he or she is lying on mat 11. Here, acupressure structures 17c and 17a are in regions 62b and 62c, respectively. In this way, some of structures 17 are preferentially located in these regions to provide desired therapeutic modalities and acupressure to desired portions of the person's body. In this example, portions of surface 62a outside of regions 62b and 62c do not include trenches 61, although in other examples they can.

FIGS. 5d and 5e are top views of another embodiment of thermal system 21 of FIG. 5a, in accordance with the present invention. In this embodiment, trenches 61 have half cylindrical shapes which extend perpendicular to a longitudinal axis of thermal system 21. It should be noted that trenches 61 can extend in other directions relative to the longitudinal axis of system 21. For example, they can extend parallel to it or at a non-zero angle, as shown in FIG. 5e.

FIG. 6 is a perspective view of an embodiment of a thermal system 70, in accordance with the present invention. Thermal system 70 includes a plurality of compartments 71a and 71b which extend through an upper surface 70a towards an opposed surface 70b. It should be noted that system 70 can have one or more compartments, but two are shown here for simplicity. Surface 70a is for receiving mat 11 and surface 70b is for engaging a support structure (not shown), such as a floor. In accordance with the invention, compartments 71a and 71b are used to store hot and cold material (not shown) to provide a desired flow of heat 13 (FIG. 1a). This feature is described in more detail above with thermal system 21. To flow heat 13 away from thermal system 70 and to conduits 12, the material can include a thermoelectric heater, hot water, heated stones, or heated metal, for example. In one example, the stones and metal are heated in hot water and then positioned in compartments 71a and 71b. In this way, the temperature of conduits 12 is increased to provide heat therapy. To flow heat 13 towards thermal system 70 and away from conduits 12, the material can include a thermoelectric cooler, cold water, ice, or cold packs, for example. In this way, the temperature of conduits 12 is decreased to provide cold therapy.

In other examples, different regions of mat 11 can be provided with different temperatures. In one example, compartment 71a is used to store a hot material and compartment 71b is used to store a cold material. If compartment 71a is in region 21c and compartment 71b is in region 21d (FIG. 4c), then heat therapy is provided to the person's head and cold therapy is provided to the person's feet. In other examples, the cold material is in compartment 71a and the hot material is in compartment 71b so that cold therapy is provided to the person's head and heat therapy is provided to the person's feet. It should be noted that thermal systems 60, 62, and 70 can replace thermal system 21 in the embodiments discussed herein.

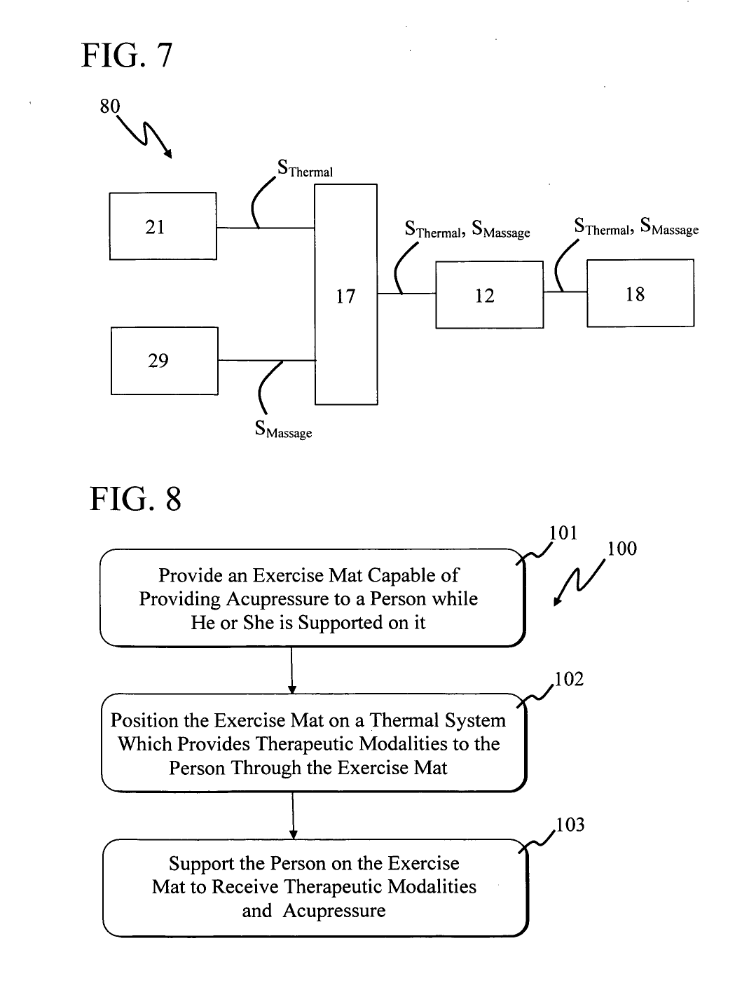

FIG. 7 is a block diagram of a system 80 which can be used to provide therapeutic modalities for the person exercising on exercise apparatus 30 as shown in FIG. 2c. In this embodiment, system 80 includes thermal system 21 which provides a signal S_{Thermal} to acupressure structures 17. System 80 also includes a massaging system 29 which provides a massaging signal S_{Massage} to acupressure structures 17. It should be noted that signal S_{Thermal} corresponds to the flow of heat, such as heat 13, and signal S_{Massage} corresponds to a vibration, which is typically used to massage the person. Massager 29 can be of many different types. One type of massager is disclosed in U.S. Pat. No. 6,077,238. This massager, or a similar one, can be modified for use in system 80 to provide signal S_{Massage}.

In one mode of operation, heat therapy is provided so that signals S_{Thermal} and S_{Massage} flow from thermal system 21 and massager 29, respectively, through acupressure structures 17 to conduits 12. Signals S_{Thermal} and S_{Massage} then flow through conduits 12 to acupressure structures 18 to the person. In another mode of operation, cold therapy is provided so that signal S_{Massage} flows as described above and signal S_{Thermal} flows from acupressure structures 18 to conduits 12. Signal S_{Thermal} then flows through acupressure structures 17 to thermal system 21.

When providing heat therapy, signal S_{Thermal} corresponds to heat flowing from thermal system 21 to structures 17 and 18, as well as conduits 12, to increase their temperature. When providing cold therapy, signal S_{Thermal} corresponds to heat flowing from acupressure structures 17 and 18, as well as conduits 12, to thermal system 21 to decrease their temperature. It should be noted that in some embodiments, thermal system 21 can be replaced with a heater for providing heat therapy only or a cooler for providing cold therapy only. It should also be noted that heater system 21 in FIG. 7 can also be replaced with heater systems 60, 62, or 70 discussed above.

FIG. 8 is a flowchart of a method 100 of exercising using an exercise apparatus, in accordance with the present invention. Method 100 includes a step 101 of providing an exercise mat. The exercise mat is capable of providing therapeutic modalities and acupressure to a person while he or she is supported on it. The exercise mat is capable of providing acupressure by having a conduit extending through it. In some embodiments, the conduit can have one or more acupressure structures coupled to it, which are preferably thermally conductive. In a step 102, the exercise mat is positioned on a thermal system, which can operate as a heater, cooler, or heater/cooler. Hence, the thermal system can provide heat, cold, and contrast therapy. In a step 103, the person exercises on the exercise mat to receive acupressure and the therapeutic modalities provided by the thermal system and exercise mat.

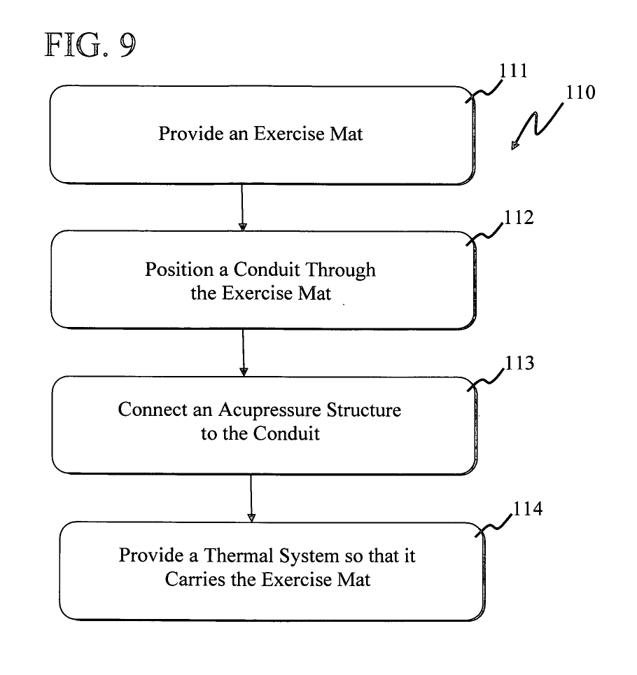

FIG. 9 is a flowchart of a method 110 of fabricating an exercise apparatus, in accordance with the present invention. Method 120 includes a step 111 of providing an exercise mat. In a step 112, a conduit is positioned through the exercise mat. The conduit is capable of flowing heat and providing a person supported on the mat with acupressure. In an optional step 113, an acupressure structure is connected to the conduit. The acupressure structure is also capable of flowing heat and providing acupressure to the person supported on the mat. In some embodiments, method 110 includes a step 114 of providing a thermal system and placing the exercise mat thereon. The thermal system is capable of flowing heat to and from the conduit and acupressure structures to provide therapeutic modalities to a person supported on the exercise mat.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

1. An apparatus, comprising:
a support structure including an exercise mat having at least one conduit extending therethrough, the conduit(s) providing acupressure to a person supported by the support structure and being capable of flowing heat to provide therapeutic modalities to the person.

2. The apparatus of claim 1, wherein the conduit(s) include a high thermal conductivity material.

3. The apparatus of claim 1, wherein the mat includes a softer material than the material included in the conduit(s).

4. The apparatus of claim 1, wherein the mat includes a material that is less thermally conductive than the material included in the conduit(s).

5. The apparatus of claim 1, wherein, in response to a force applied thereto, the support structure has a thickness in a region that includes the conduit(s) and a smaller thickness in a region away from the conduit(s).

6. The apparatus of claim 1, further including a thermal system which flows heat through the conduit(s).

7. The apparatus of claim 1, wherein the thermal conductivity of the material in the conduit(s) is between about 2 W/m×K and 450 W/m×K.

8. The apparatus of claim 1, wherein the thermal conductivity of the material in the exercise mat is between about 0.001 W/m×K and 0.7 W/m×K.

9. The apparatus of claim 1, wherein the thermal conductivity of the conduit(s) is more than three times the thermal conductivity of the exercise mat.

10. The apparatus of claim 1, further including a massager which flows a massage signal through the conduit(s).

11. Apparatus, comprising:
a thermal system;
an exercise mat carried by the thermal system; and
a first acupressure structure positioned adjacent to a surface of the exercise mat proximate to the thermal system or an opposed surface of the exercise mat away from the thermal system, the first acupressure structure including a material with a high thermal conductivity.

12. The apparatus of claim 11, further including a conduit connected to the first acupressure structure and extending through the exercise mat.

13. The apparatus of claim 12, further including a second acupressure structure connected to the conduit opposite the first acupressure structure.

14. The apparatus of claim 13, further including a plurality of conduits which extend through the exercise mat, at least one conduit having opposed ends connected to first and second acupressure structures.

15. The apparatus of claim 11, wherein the thermal system and exercise mat are integrated together as a single piece.

16. The apparatus of claim 15, wherein the thermal system and exercise mat are integrated together to form a cavity therebetween.

17. The apparatus of claim 16, wherein the first acupressure structure is housed within the cavity.

18. The apparatus of claim 11, wherein the thermal system controls the temperature of the first acupressure structure.

19. The system of claim 11, further including at least one trench structure which extends through a surface of the thermal system facing the exercise mat.

20. The system of claim 11, wherein the exercise mat includes at least one of rubber and plastic.

21. A method of exercising, comprising:
providing a support structure which provides acupressure to a person while the person is supported on it, the support structure including an exercise mat;
positioning the support structure on a thermal system which provides therapeutic modalities to the person through the support structure; and
supporting the person on the support structure so that the person receives acupressure and therapeutic modalities.

22. The method of claim 21, further including positioning one or more acupressure structures adjacent to the exercise mat.

23. The method of claim 21, further including positioning one or more conduits through the exercise mat.

24. The method of claim 23, further including flowing heat between the thermal system and conduit.

25. The method of claim 21, further including positioning a conduit through the exercise mat and connecting one or more acupressure structures to it.

26. The method of claim 21, further including having the person exercise while supported by the support structure.

27. A method of fabricating an exercise apparatus, comprising:
providing an exercise mat;
positioning a conduit through the exercise mat; and
connecting a first acupressure structure to the conduit.

28. The method of claim 27, wherein the conduit and first acupressure structure include at least one of stone and metal and the exercise mat includes at least one of rubber and plastic.

29. The method of claim 27, wherein the conduit includes a high thermal conductivity material and the exercise mat includes a low thermal conductivity material.

30. The method of claim 27, further including connecting a second acupressure structure to the conduit, the first and second acupressure structures being connected to opposed ends of the conduit.

31. The method of claim 27, further including providing a thermal system which carries the exercise mat.

32. The method of claim 27, wherein the acupressure structure and conduit are threadingly engaged to each other.
